(19) United States
(12) Patent Application Publication
(10) Pub. No.: US 2010/0093657 A1
(43) Pub. Date: Apr. 15, 2010

(54) LOW GLYCEMIC MIXTURES

(76) Inventors: Jörg Kowalczyk, Eisenberg-Steinborn (DE); Stephan Hausmanns, Wiesbaden (DE)

Correspondence Address: HARNESS, DICKEY & PIERCE, P.L.C., P.O. BOX 828, BLOOMFIELD HILLS, MI, 48303, US

(21) Appl. No.: 12/528,053

(22) PCT Filed: Feb. 22, 2008
(86) PCT No.: PCT/EP08/01394

§ 371 Date: Aug. 20, 2009

(30) Foreign Application Priority Data
Feb. 23, 2007 (DE) 10 2007 009 029.5

(51) Int. Cl. A61K 31/7016; A23L 1/22; A23L 1/236; A23L 1/29; A23L 1/30; A23D 7/015; A61P 3/00; C07H 3/04

(52) U.S. Cl. 514/53; 426/602; 426/648; 426/61; 426/71; 426/548; 426/72; 426/74; 536/123.13

(57) ABSTRACT

The present invention relates to a food compound comprising an emulsion containing triglyceride oils as well as a low-glycemic saccharose isomer.

The present invention relates to food compositions to control weight and/or appetite, comprising at least two components, each of which showing at least one nutritionally positive-working functionality, with the first component showing an appetite-depressant function and the second component showing a low-glycemic function, as well as the use of low-glycemic food components in food compositions.

Modern foods must meet various requirements these days. In addition to their primary function as a source of energy, particularly a health-preserving and health-promoting function has also been attributed to them more recently. Food should taste good, guarantee the necessary supply of energy, but also counteract undesired weight gain with its associated negative health consequences at the same time.

Probiotic yoghurt is known from U.S. Pat. No. 6,025,008, which contains refined fish oil along with a sweetener, for example isomaltulose, maltose, trehalose or maltitol. Yoghurt is nutritionally beneficial, since it may serve as prophylaxis against, and as a treatment of cardiovascular diseases due to its content of unsaturated fatty acids. In this connection, the sweeteners employed mask the undesired fishy odor in many cases.

WO 03/022288 A1 shows food compositions that contain lipids and carbohydrates. Palatinose or trehalulose can be used as a carbohydrate in this regard. The food compositions described there prove to be nutritionally sensible for patients suffering from diabetes, as well as to prevent obesity.

From WO 2004/057982 emerges the use of certain triacylglycerols in foods to achieve a sensation of satiety.

However, an additional need for foods that involve beneficial nutritional and technological properties, such as for example increased stability, still exists. It is particularly desirable to provide a storage-stable food composition that tastes good and contains food components necessary to keep consumers healthy, on the one hand, but on the other hand does not entice consumers to excessive consumption, and thus ultimately to obesity, especially since such food composition can be produced relatively simply and cost-effectively.

The present invention solves its underlying technical problem by providing a food composition to control weight and/or appetite, comprising at least two components, each of which showing at least one nutritionally positive-working functionality, with the first component showing an appetite-depressant function and the second component showing a low-glycemic function, and with both components present in the food composition in a quantity that makes it possible to develop their nutritionally positive functionality. The invention particularly solves its underlying problem by providing such a food composition, with the first component comprising an emulsion, particularly an oil-in-water emulsion, with triacylglycerol or triglyceride concentrations, respectively. In a particularly preferred embodiment the triglyceride oils in the oil phase are embodied such that they exist in solid form between room temperature and the body temperature of a healthy human. Preferably, such triglycerides are palm oil or cocoa butter, or fractions thereof. In a preferred embodiment they should be available in solid form between room temperature and the consumer's body temperature. Preferably, said triglyceride oils exhibit at least 90, preferably at least 95 percent by weight, especially preferred at least 99 percent by weight of triglycerides (relative to the total weight of the triglyceride oil). Preferably, such triglycerides are glycerol esters of linoleic acid, oleic acid, palmitic or stearic acid.

In addition to the triglyceride oils in the emulsion, the emulsion utilized according to the invention containing triglyceride oils also features at least one emulsifying agent, which preferably is compatible with food, and which keeps the triglycerides in emulsion in the aqueous phase. In a particularly preferred embodiment this involves a galactolipid-based emulsifying agent, for example mono or digalactosyl diglyceride or lecithin. The quantity and type of emulsifying agent according to the invention must be utilized such that the triglyceride oils in the emulsion can be kept in emulsion, in particular between room temperature and the consumer's body temperature.

In a particularly preferred embodiment of the invention the galactolipid is oat oil, particularly fractionated oat oil.

A further preferred embodiment provides that the oil phase of the emulsion contains 80 to 99 percent by weight of triglyceride oils and 1 to 20 percent by weight of an emulsifying agent that is compatible with food (in each case relative to the weight of the oil phase).

A further embodiment may provide that the triglycerides are present in conjunction with coconut oil or palm kernel oil

A particularly preferred embodiment may provide that the aqueous component of the emulsion is water. In addition, the latter may also be provided with flavor additives, artificial coloring, flavoring agents, sweeteners, thickening agents, preserving agents, antioxidants, minerals, trace elements, etc.

In a particularly preferred embodiment of the invention the aqueous phase of the emulsion itself may contain the second component of the food composition according to the invention, i.e. the low-glycemic saccharose isomer, namely particularly isomaltulose and/or leucrose.

A particularly preferred embodiment provides that the total fat content of the emulsion amounts to 40 to 50 percent by weight (relative to the total weight of the emulsion).

In the context of the invention a low-glycemic saccharose isomer means an isomer, i.e. a structural isomer of saccharose, that works as a low-glycemic carbohydrate in the human body, namely that results in a relatively low respiratory quotient following consumption. The respiratory quotient reflects the CO₂/O₂ ratio in the breathable air, and represents a measure for the nutrients burnt. Pure carbohydrate burning results in a respiratory quotient of 1, while pure fat burning leads to a respiratory quotient of 0.7. Low-glycemic carbohydrates thus passively support the utilization of fat to produce energy in the body, since they don't allow the insulin level to rise too much, and thus favor fat oxidation.

In the context of the invention low-glycemic saccharose isomers especially refer to leucrose and isomaltulose. The term “low-glycemic saccharose isomer” within the context of the present invention especially means that a) isomaltulose or b) leucrose or c) leucrose and isomaltulose, preferably in a ratio of 1:99 percent by weight to 99:1 percent by weight, preferably 30:70 percent by weight to 70:30 percent by weight, are utilized as a low-glycemic carbohydrate in the food composition according to the invention (in each case relative to the total dry substance of the two isomers).

The first component of the food composition according to the invention causes the consumer to experience a heightened, quickly beginning and prolonged sensation of satiety, and hence features an appetite-depressant and thus weight-controlling functionality.

The combination of the appetite-depressant first component and the second component, particularly the low-glycemic saccharose isomer, especially isomaltulose and/or leucrose, surprisingly results in a particularly storage-stable mixture, especially with particularly positive nutritional functionalities. These nutritionally positive functionalities of the first component include the consumer's quickly beginning satiety caused by the food composition according to the invention, which at an early stage leads to a sustained, long-lasting subsiding sensation of hunger, and thus serves as prophylaxis and treatment of obesity, the control of calorie and fat intake, and consequently ultimately also prevents cardiovascular diseases and diabetes. The nutritionally positive functionalities of the second component include its characteristic to release glucose in the consumer's digestive tract at a reduced speed, its characteristic not to cause the blood glucose level to rise too rapidly, and its low-glycemic property, and thus lead to a lower increase in the insulin level, with the consequence of favoring the oxidation of fat. On the other hand, it was surprising to find that the stability of the fatty first component in a food composition was significantly increased in the presence of at least one low-glycemic saccharose isomer, particularly of isomaltulose, leucrose, or a combination of isomaltulose and leucrose, especially the stability of a surface-enlarged flake or aggregate form, in which the components of the composition according to the invention are present in a preferred embodiment. Finally, due to the presence of the second component, improved processability of the first component emerges in the course of the production of various foods.

In a particularly preferred embodiment the present invention relates to an aforementioned food composition, with said food composition containing 1 to 50 percent by weight of the first component, preferably 2 to 50 percent by weight, preferably 2 to 15 percent by weight, particularly 3 to 10 percent by weight, preferably 3 to 50 percent by weight, particularly 4 to 40 percent by weight and particularly 5 to 30 percent by weight of the first component (in each case relative to the total dry substance of the food composition).

In a further preferred embodiment the invention relates to an aforementioned food composition, with said food composition containing preferably 1 to 50 percent by weight of a low-glycemic saccharose isomer, particularly 2 to 50 percent by weight, preferably 2 to 15 percent by weight, particularly 3 to 10 percent by weight, particularly 3 to 50 percent by weight, particularly 4 to 40 percent by weight or 5 to 30 percent by weight of a low-glycemic saccharose isomer (in each case relative to the total dry substance of the food composition).

The present invention also particularly relates to one of the aforementioned food compositions, comprising 1, 2, 3, 4, 5, 6, 7, 8, 9 or 10 percent by weight to 10, 15, 20, 30, 40 or 50 percent by weight of the first component and 1, 2, 3, 4, 5, 6, 7, 8, 9 or 10 percent by weight to 10, 15, 20, 30, 40 or 50 percent by weight of low-glycemic saccharose isomer (in each case relative to the total dry substance of the food composition).

In a further preferred embodiment the food composition of the present invention—in addition to the first component and at least the one low-glycemic saccharose isomer—comprises at least one, preferably several additive(s), particularly in a quantity adding up to 100 percent by weight of total dry matter content, for example in a quantity from 1 to 99 percent by weight, preferably 40 to 90 percent by weight, preferably 2 to 85 percent by weight, 3 to 70 percent by weight, 5 to 60 percent by weight, 10 to 50 percent by weight, 20 to 40 percent by weight, 30 to 80 percent by weight, 40 to 70 percent by weight, 45 to 60 percent by weight or 25 to 38 percent by weight, relative to the total dry substance of the food composition.

An additive refers to such substances that influence in particular the food composition's appearance, taste, organoleptics, nutritional value, nutritional properties, processability, shelf life or readiness for use.

A further preferred embodiment provides that at least one additive constitutes a prebiotic, a probiotic, a supplement, a fatty component, a milk product or a sweetening agent.

In the context of the present invention the term “sweetening agent” refers to substances that possess sweetening power and are added, for example, to foods or beverages to produce a sweetening taste. In the context of the present invention “sweetening agents” are divided into “sugars,” such as isomaltulose, saccharose, glucose or fructose, that provide body and sweetening power, as well as “sweetening agents,” namely substances that are not sugars, but still possess sweetening power, which in turn are further subdivided into “sugar substitutes,” namely sweetening agents that possess body and physiological calorific value in addition to its sweetening power (body-giving sweeteners), and “high-intensity sweeteners,” namely substances that generally have a very high sweetening power, but no body, and generally possess no or only little calorific value. High-intensity sweeteners are for example cyclamate, acesulfame H, aspartame or sucralose.

Thus, in a particularly preferred embodiment the sweetening agent is a sugar, a high-intensity sweetener or a sugar substitute.

In a further preferred embodiment the high-intensity sweetener is selected from the group that is comprised of sucralose, sodium cyclamate, acesulfame K, neohesperidin dihydrochalcone, glycyrrhizin, stevioside, monellin, thaumatin, aspartame, dulcin, saccharin, naringin dihydrochalcone, neotame, and a mixture of two or several thereof. In a further preferred embodiment the sugar substitute is selected from the group consisting of isomalt, 1,1-GPM (1-O-α-D-Glucopyranosyl-D-mannitol), 1,6-GPS (6-O-α-D-Glucopyranosyl-D-sorbit), 1,1-GPS (1-O-α-D-Glucopyranosyl-D-sorbitol), maltodextrins, lactitol, maltitol, erythritol, xylitol, mannitol, sorbitol, maltitol syrup, hydrated and non-hydrated starch hydrolysates and a mixture of two or several thereof.

In a further preferred embodiment at least the one additive is a sugar, particularly saccharose, glucose, fructose, lactose, maltose or a mixture of two or several thereof.

Naturally, the invention also relates to food compositions comprising the first component and at least the one low-glycemic saccharose isomer, with said low-glycemic saccharose isomer being the one and only body-giving sweetening agent that occurs in the food composition. In a further preferred embodiment the invention relates to an aforementioned food composition containing the first component and at least the one low-glycemic saccharose isomer, with said low-glycemic saccharose isomer being the one and only sugar agent that occurs in food composition. In a further preferred embodiment the invention relates to a food composition comprising the first component and at least the one low-glycemic saccharose isomer, with said low-glycemic saccharose isomer being the one and only sweetening agent that occurs in food composition.

In particular, the invention relates to a food composition comprising, or consisting of, the first component and at least the one low-glycemic saccharose isomer, with the food composition optionally being suited for diabetics or being dietetic.

In a further preferred embodiment the present invention relates to an available food composition containing the first component and at least the one low-glycemic saccharose isomer, with said food composition being free of saccharose, free of lactose, free of fructose, free of sorbitol, free of xylitol, free of mannitol, or free of one or several or all of the aforementioned sugars or sugar alcohols.

In a further preferred embodiment the present invention relates to a food composition containing the first component and at least the one low-glycemic saccharose isomer, with said composition containing as an additive a prebiotic, particularly inulin, oligofructose, galacto-oligosaccharide.

In a further preferred embodiment the present invention relates to a food composition containing the first component, an additive and at least the one low-glycemic saccharose isomer, with at least the one additive being a milk product, particularly a lactose-free milk product, for example skim milk powder, whole milk powder, lactose-free skim milk powder, lactose-free whole milk powder, whey solids, or a mixture of two or several thereof.

In a further preferred embodiment the present invention relates to a food composition containing the first component, an additive and at least the one low-glycemic saccharose isomer, with at least the one additive being a supplement selected from the group consisting of malt extract, flavoring agents, artificial coloring, preserving agents, flavors, release agents, flavor additives, flow agents, mineral nutrients like sodium and calcium, particularly salts such as sodium chloride, vitamins, folic acid, emulsifiers, dietary fibers, lecithin, omega-3-fatty acids, medium chain length triglycerides, phythoestrogens and ascorbic acid salts.

In a further preferred embodiment the invention relates to a food composition containing the first component, an additive and at least the one low-glycemic saccharose isomer, with probiotic, for example lactobacillus or bifidobacteria, being present as an additive.

In a further preferred embodiment the food composition of the present invention is a product, embodied as food, a stimulant or a pharmaceutical, preferably as a dietetic food or stimulant.

According to the invention the food, stimulant or pharmaceutical preferably comprises 1 to 99 percent by weight, 2 to 99 percent by weight, particularly 20 to 70 percent by weight, preferably 30 to 60 percent by weight, more preferably 40 to 55 percent by weight (relative to the total dry substance of the food, stimulant or pharmaceutical) of the first and second component. In a particular embodiment the food, stimulant or pharmaceutical is free of glucose, fructose, lactose, saccharose, sorbitol, xylitol and/or mannitol. According to the invention, however, it may also contain glucose, fructose, saccharose and/or other sweetening agents.

In the context of the present invention the term “food” predominantly refers to products or mixtures of substances in solid, semi-solid, liquid, dissolved or suspended form serving as human nutrition that are mainly intended to be consumed by humans in unchanged, prepared or processed condition. Besides their natural components foods may contain additional components of natural or synthetic origin. Foods may be available in solid form as well as in liquid form. Substances or mixtures of substances in solid, semi-solid, liquid, dissolved or suspended form serving the enjoyment of the human or animal body during consumption are predominantly referred to as “stimulants.”

In a preferred embodiment of the invention the foods according to the invention involve milk products or dairy products, for example cheese, butter, yoghurt, ice cream, drinking yoghurt, kefir, curd cheese, curdled milk, buttermilk, cream, condensed milk, powdered milk, whey, mixed milk, half-fat butter, mixed dairy, milk sugar, milk protein and milk fat products. In a further preferred embodiment of the invention the foods according to the invention involve baked goods, particularly bread, small baked goods, fine baked goods, long-life bakery products. In further embodiments of the invention the foods according to the invention involve spreads, margarine products and shortenings, as well as instant products and brew products. A food within the meaning of this invention can also be a beverage powder, for example an instant beverage powder, for example a cocoa, tea or coffee product beverage powder.

In a particularly preferred embodiment of the present invention the food comprises a liquid food, namely a beverage, particularly a water-based or milk-based beverage. The beverages may be alcoholic or non-alcoholic. In a preferred embodiment the beverages may be suitable for diabetics. In a particularly preferred embodiment the food composition may be a beverage, for example a milk beverage, a milk-mix beverage, a sports beverage, an energy beverage, an enteral formulation, a refreshment beverage, a beverage containing Cola, or the like. The term stimulants, for example, refers to solid or semi-solid confectionary, particularly chocolate products, hard caramels, soft caramels, fondant products, jelly products, licorice, foam sugar goods, coconut flakes, candy-coated sticks, lozenges, candied fruit, brittle, nougat products, ice cups, marzipan, chewing gum, cereal bars, as well as ice cream.

In the context of the present invention “dietetic foods or beverages” refer to foods or beverages intended to serve a specific nutritional purpose, that they cause the supply of certain nutrients or other nutritionally acting substances in a certain proportion or in a certain condition. Dietetic foods or beverages significantly differ from comparable foods or beverages through their composition or their characteristics. Dietetic foods can be utilized in cases, in which certain dietary requirements must be fulfilled due to illnesses, functional disorders or allergic reactions against individual foods or their ingredients. Dietetic foods may be present in solid, semi-solid and liquid forms (beverage).

The invention also relates to methods for the production of the aforementioned food compositions. Hence, the invention provides for the provision of a method to produce the aforementioned food compositions, in which the first component and the second component, particularly at least the one low-glycemic saccharose isomer, is furnished, and mixed with one another in the desired ratio, if necessary, together with at least the one additive.

In a further preferred embodiment the present invention involves the use of at least the one low-glycemic saccharose isomer, particularly isomaltulose and/or leucrose, as a low-glycemic component in a food, stimulant or pharmaceutical that contains an emulsion with triglyceride oils to control weight, appetite, the intake of fat, the intake of calories, to improve the utilization of fat and/or calories, as prophylaxis against, or treatment of obesity, adiposity, diabetes, cardiovascular diseases, or the like.

The invention also relates to the use of at least one low-glycemic saccharose isomer, particularly isomaltulose and/or leucrose, in a food, stimulant or pharmaceutical containing an emulsion with triglyceride oils to increase the stability of the emulsion in the food, stimulant or pharmaceutical.

Further advantageous embodiments of the invention result from the subclaims.

The invention is described in greater detail based on the enclosed example.

### EXAMPLE

To demonstrate the filling effect of an isomaltulose (and leucrose) palm kernel oil mixture, the improved stability, and thus an improved property profile, is shown when using isomaltulose or leucrose as a stability-improving agent on the basis of in-vitro tests.

As raw material palm kernel oil was used as an example for the triglyceride, and lecithin as an emulsifying agent. Tests, in which only these two components were used, as well as tests with different concentrations of carbohydrate additives, were conducted. Besides isomaltulose and leucrose, saccharose was also tested for control purposes.

The emulsions were produced by high-pressure emulsification. After an initial screening it was possible to demonstrate that visually stable emulsions were obtained already at a pressure of 700 bar. The high-pressure emulsification was ultimately carried out at pressures from 790 to 810 bar.

10 g of the products obtained through high-pressure emulsification were then introduced into 200 ml of a synthetic gastric juice (for 1,000 ml 3.25 g pepsin, 2.02 g sodium chloride are dissolved in 500 ml water, adjusted with 1 M HCl to pH 1, and then filled with distilled water) at 37° C. by stirring, and the stability or the demulsibility properties (release from oil) were observed in relation to the time. To be able to derive an impact in terms of the filling effect also from a physical-chemical perspective, the generated dispersions should be as voluminous as possible, namely show a large surface, and they should also be stable, preferably up to one hour. This could clearly be demonstrated for the two reducing disaccharides isomaltulose and leucrose.

Formulations of the test emulsions A-F (information in % by weight):

| Test | A | B | C | D | E |
|---|---|---|---|---|---|
| Palm kernel | 40 | 40 | 40 | 40 | 40 |
| oil | | | | | |
| Lecithin | 2 | 2 | 2 | 2 | 2 |
| Saccharose | — | 3 | — | — | — |
| Isomaltulose | — | — | 2 | 6 | — |
| Leucrose | — | — | — | — | 2 |
| Distilled wa- | 58 | 55 | 56 | 55 | 56 |
| ter | | | | | |

Results following dispersion in the synthetic gastric juice:

The following grading was used to assess the stability:

++ voluminous stable flakes at the surface
+ slightly aggregated flakes at the surface, no demulsibility
− slightly aggregated flakes at the surface, beginning demulsibility (oil droplets)
−− aggregated flakes, clear demulsibility (oil separation)

| Time in min- | | | | | |
|---|---|---|---|---|---|
| utes | A | B | C | D | E |
| 1-2 | ++ | ++ | ++ | ++ | ++ |
| 15 | ++ | ++ | ++ | ++ | ++ |
| 30 | − | + | ++ | ++ | ++ |
| 45 | −− | − | ++ | ++ | ++ |
| 60 | | −− | + | + | + |

The results demonstrate that the use of isomaltulose and leucrose in the palm kernel oil/lecithin mixtures has a clearly positive impact on the stability, particularly, that it causes improved, namely increased stability of the forming surface-enlarged flaked particles.

1-29. (canceled)

30. A food composition to control weight and/or appetite, comprising at least two components, each of the at least two components having at least one nutritionally positive-working functionality, wherein the first component has an appetite-depressant function and the second component has a low-glycemic function, both components present in the food composition in a quantity that makes it possible to develop their nutritionally positive functionality, the first component being an emulsion containing triglyceride oils, with the triglyceride oils selected from a group including palm oil and cocoa butter, and the second component being selected from a group including isomaltulose and leucrose, with the emulsion being an oil-in-water emulsion comprised of triglyceride oils with a food-compatible emulsifying agent in an aqueous solution, with said food-compatible emulsifying agent being selected from a group including a galactolipid and lecithin.

31. The food composition in accordance with claim 30, wherein the triglyceride oils are fractionated palm oil.

32. The food composition in accordance with claim 30, wherein the galactolipid is oat oil.

33. The food composition in accordance with claim 32, wherein the oat oil is fractionated oat oil.

34. The food composition in accordance with claim 30, wherein the oil phase of the emulsion contains 80 to 99 percent by weight of triglyceride oils and 1 to 20 percent by weight of a food-compatible emulsifying agent in each case relative to the oil phase.

35. The food composition in accordance with claim 30, wherein at least 90 percent by weight of the triglyceride oils are comprised of triglycerides.

36. The food composition in accordance with claim 30, wherein the triglyceride oils are present in conjunction with coconut or palm kernel oil.

37. The food composition in accordance with claim 30, wherein a total fat content of the emulsion amounts to 40 to 50 percent by weight relative to the total weight of the emulsion.

38. The food composition in accordance with claim 30, wherein the food composition contains 1 to 50 percent by weight of the first component relative to the total dry substance of the food composition.

39. The food composition in accordance with claim 30, wherein the food composition contains 1 to 50 percent by weight of the second component relative to the total dry substance of the food composition.

40. The food composition in accordance with claim 38, wherein the food composition contains 5 to 30 percent by weight of the first component relative to the total dry substance of the food composition.

41. The food composition in accordance with claim 39, wherein the food composition contains 5 to 30 percent by weight of the second component relative to the total dry substance of the food composition.

42. The food composition in accordance with claim 30, wherein the food composition is a solid, semi-solid or liquid food, food product or pharmaceutical.

43. The food composition in accordance with claim 30, wherein the food composition contains 3 to 50 percent by weight of the first component and 3 to 50 percent by weight of the second component in each case relative to the total dry substance of the food composition.

44. The food composition in accordance with claim 30, wherein the food composition contains at least one additive.

45. The food composition in accordance with claim 44, wherein at least the one additive is selected from a group including a prebiotic, a probiotic, a supplement, a fatty component, a milk product and a sweetening agent.

46. The food composition in accordance with claim 45, wherein the sweetening agent is selected from a group including a sugar, a high-intensity sweetener and a sugar substitute.

47. The food composition in accordance with 45, wherein the prebiotic is selected from a group including inulin, oligofructose, galacto-oligosaccharides and mixtures thereof.

48. The food composition in accordance with claim 46, wherein the high-intensity sweetener is selected from a group including sucralose, sodium cyclamate, acesulfame K, neohesperidin dihydrochalcone, glycyrrhizin, stevioside, monellin, thaumatin, aspartame, dulcin, saccharin, naringin dihydrochalcone, neotame, and combinations thereof.

49. The food composition in accordance with claim 46, wherein the sugar substitute is selected from a group including isomalt, 1,1-GPM (1-O-α-D-Glucopyranosyl-D-mannite), 1,6-GPS (6-O-α-D-Glucopyranosyl-D-sorbit), 1,1-GPS (1-O-α-D-Glucopyranosyl-D-sorbitol), maltodextrins, lactitol, maltitol, erythritol, xylitol, mannitol, sorbitol, maltitol syrup, hydrated and non-hydrated starch hydrolysates, and combinations thereof.

50. The food composition in accordance with claim 44, wherein the at least one additive is a sugar.

51. The food composition in accordance with claim 50, wherein the sugar is selected from a group including saccharose, glucose, fructose, lactose, maltose, and combinations thereof.

52. The food composition in accordance with claim 44, wherein the at least one additive is a milk product.

53. The food composition in accordance with claim 52, wherein the milk product is a lactose-free milk product.

54. The food composition in accordance with claim 52, wherein the milk product is selected from a group including skim milk powder, whole milk powder, lactose-free skim milk powder, lactose-free whole milk powder, whey solid, and combinations thereof.

55. The food composition in accordance with claim 44, wherein the at least one additive is a supplement selected from a group including malt extract, flavoring agents, artificial coloring, preserving agents, flavors, release agents, flavor additives, flow agents, mineral nutrients like sodium and calcium, particularly salts such as sodium chloride, vitamins, folic acid, emulsifiers, dietary fibers, lecithin, omega-3-fatty acids, medium chain length triglycerides, phythoestrogens and ascorbic acid salts.

56. The food composition in accordance with claim 45, wherein the probiotic is a lactobacillus or bifidobacteria.

57. The food composition in accordance with claim 45, wherein isomaltulose or leucrose is the one and only body-giving sweetening agent that occurs in the food composition.

58. The food composition in accordance with claim 46, wherein isomaltulose or leucrose is the one and only sugar that occurs in the food composition.

59. The food composition in accordance with claim 45, wherein isomaltulose or leucrose is the one and only sweetening agent that occurs in the food composition.

60. Use of isomaltulose or leucrose as a low-glycemic component to control weight, appetite, the intake of fat, the intake of calories, to improve the utilization of fat and/or calories, as prophylaxis against, or treatment of obesity, adiposity, diabetes or cardiovascular diseases, in a food, stimulant or pharmaceutical that contains an emulsion with triglyceride oils.

61. Use of isomaltulose and/or leucrose in a food, stimulant or pharmaceutical containing an emulsion with triglyceride oils to increase the stability of the emulsion in the food, stimulant or pharmaceutical.
